(19) United States
(12) Patent Application Publication
(10) Pub. No.: US 2025/0128094 A1
(43) Pub. Date: Apr. 24, 2025

(54) RADIOTHERAPY SYSTEM AND METHOD FOR CONTROLLING THE SAME

(71) Applicant: OUR UNITED CORPORATION, Xi'an (CN)

(72) Inventors: Jinsheng LI, Xi'an (CN); Zhongyuan ZHANG, Xi'an (CN); Hong CHENG, Xi'an (CN)

(21) Appl. No.: 18/920,598

(22) Filed: Oct. 18, 2024

(30) Foreign Application Priority Data
Oct. 20, 2023 (CN) 202311369439.7

(51) Int. Cl. A61N 5/10

(52) U.S. Cl. CPC A61N 5/1081; A61N 5/1045; A61N 5/1067

(57) ABSTRACT

The present disclosure discloses a radiotherapy system, including: a radiation source configured to emit therapeutic rays; a treatment couch configured to carry a patient, the radiation source being capable of rotating around a longitudinal direction of the patient; and a processor configured to control the radiation source to continuously rotate for a radian of greater than 360 degrees in a first direction from a first position around the longitudinal direction of the patient to a second position.

### CROSS-REFERENCE TO RELATED APPLICATION

This application is based on and claims priority to Chinese Patient application Ser. No. 202311369439.7, filed on Oct. 20, 2023, the disclosure of which is herein incorporated by reference in its entirety.

### TECHNICAL FIELD

The present disclosure relates to the field of radiotherapy technologies, and in particular, relates to a radiotherapy system and a method for controlling the same.

### BACKGROUND

Radiotherapy is a common way to treat tumors, and it can kill tumor lesions by using high-energy rays generated by a radiotherapy device.

Generally, when radiotherapy is performed for a patient's tumor, a treatment plan is first formulated according to the condition of the patient's tumor, and then the radiotherapy device applies a desired radiation dose to the patient's tumor according to the treatment plan, so as to implement the treatment of the patient's tumor.

### SUMMARY

Embodiments of the present disclosure provide a radiotherapy system and a method for controlling the same, which can control a radiation source to continuously rotate in the same direction for a radian of more than 360 degrees around a longitudinal direction of a patient, achieving high radiotherapy efficiency.

According to a first aspect, the present disclosure provides a radiotherapy system. The system includes: a radiation source, configured to emit therapeutic rays; a treatment couch, configured to carry a patient; the radiation source being capable of rotating around a longitudinal direction of the patient; and a processor, configured to control the radiation source to continuously rotate for a radian of greater than 360 degrees in a first direction from a first position around the longitudinal direction of the patient to a second position.

According to another aspect, the present disclosure provides a radiotherapy system. The system includes: a radiation source, configured to emit therapeutic rays; a treatment couch, configured to carry a patient; the radiation source being capable of rotating around a longitudinal direction of the patient; a multi-leaf collimator, configured to perform beam shaping on the therapeutic rays emitted from the radiation source; and a processor, configured to control the radiation source to continuously rotate for a radian of greater than 360 degrees in a first direction from a first position around the longitudinal direction of the patient to a second position; and control the radiation source to deliver a radiation to the patient during the continuous rotational movement of the radiation source for greater than 360 degrees around the longitudinal direction of the patient; and also control leaves of the multi-leaf collimator to continuously move.

According to another aspect, the present disclosure provides a method for controlling a radiotherapy system, which is applied to a processor of the radiotherapy system according to the first aspect, and includes: controlling the radiation source to continuously rotate for the radian of greater than 360 degrees in the first direction from the first position around the longitudinal direction of the patient to the second position.

According to yet another aspect, the present disclosure provides a method for controlling a radiotherapy system, which is applied to a processor of the radiotherapy system, wherein the radiotherapy system includes: a radiation source, configured to emit therapeutic rays; a treatment couch, configured to carry a patient; the radiation source being capable of rotating around a longitudinal direction of the patient; a multi-leaf collimator, configured to perform beam shaping on the therapeutic rays emitted from the radiation source; and a processor, configured to control the radiation source and the multi-leaf collimator; and the method includes: controlling the radiation source to continuously rotate for a radian of greater than 360 degrees in a first direction from a first position around the longitudinal direction of the patient to a second position; controlling the radiation source to deliver a radiation to the patient during the continuous rotational movement of the radiation source for greater than 360 degrees around the longitudinal direction of the patient; and also controlling leaves of the multi-leaf collimator to continuously move.

### BRIEF DESCRIPTION OF DRAWINGS

To describe the technical solutions in the embodiments of the present disclosure more clearly, the following briefly introduces the accompanying drawings required for describing the embodiments. Apparently, the accompanying drawings in the following description show merely some embodiments of the present disclosure, and a person of ordinary skill in the art may still derive other drawings from these accompanying drawings without creative efforts.

FIG. 1 is a schematic diagram of a radiotherapy system according to some embodiments of the present disclosure;

FIG. 2 is a schematic diagram of an embodiment in which a processor of a radiotherapy system controls a radiation source to move according to some embodiments of the present disclosure;

FIG. 3 is a schematic structural diagram of a radiation source and a multi-leaf collimator in a radiotherapy system according to some embodiments of the present disclosure; and

FIG. 4 is a flowchart of a method for controlling a radiotherapy system according to some embodiments of the present disclosure.

### DETAILED DESCRIPTION

The technical solutions in the embodiments of the present disclosure will be described clearly and completely below in conjunction with accompanying drawings in the embodiments of the present disclosure. In addition, the described embodiments are merely some embodiments, rather than all embodiments, of the present disclosure. Based on the embodiments of the present disclosure, all other embodiments derived by a person of ordinary skill in the art without creative efforts shall fall within the protection scope of the present disclosure.

It should be noted in the description of the present disclosure that the terms “first” and “second” are only for the purpose of description and should not be construed as indicating or implying relative importance or implicitly indicating the number of technical features indicated. Thus, the features defined by the terms “first” and “second” may include one or more of the features either explicitly or implicitly. In the present disclosure, the terms herein such as “an embodiment,” “some embodiments,” “an exemplary embodiment,” “an example,” “a specific example,” “some examples,” and “exemplary” are intended to indicate that particular features, structures, materials or characteristics described in association with the embodiments or examples are included in at least one embodiment or example of the present disclosure. The schematic description of the above terms does not necessarily refer to the same embodiment or example. Furthermore, the described particular features, structures, materials, or characteristics may be included in any one or more embodiments or examples properly. Any embodiment described in the present disclosure as “exemplarily” should not be construed as preferred or advantageous over other embodiments. Accordingly, the present disclosure will not be limited to these embodiments shown herein, but will conform to the widest range consistent with the principles and features disclosed in the present disclosure.

In general, when a radiation dose is applied to a patient, the patient lies on his/her back on a treatment couch in a flat position, and the treatment head of a radiotherapy device rotates around a longitudinal direction of the patient under the drive of a rotating gantry to deliver a desired radiation dose to a patient's tumor.

In related art, in a radiotherapy system, the rotating gantry drives the treatment head that can emit a therapeutic beam to rotate around the longitudinal direction of the patient, such that the desired radiation dose is applied to the patient's tumor. Therefore, for this radiotherapy system, the rotation angle of the rotating gantry is limited within 360 degrees due to a large-volume power transmission cable coupled to the rotating gantry. When arc radiotherapy needs to be performed from certain positions of the patient (e.g., back), due to the limited rotation of the rotating gantry, it is necessary to divide an arc into two segments to perform arc radiotherapy separately. In addition, in the case that a patient needs to undergo full-circle are radiotherapy, it can be achieved by round-trip arc radiotherapy. Segmented are radiotherapy or round-trip arc radiotherapy can lead to an increase in the radiotherapy duration and low efficiency of radiotherapy.

In view of the above, the present disclosure provides a radiotherapy system, which can realize the rotation of the radiation source in the same direction for a radian of more than 360 degrees around the longitudinal direction of the patient, so as to avoid segmented arc radiotherapy and round-trip arc radiotherapy, thereby achieving continuously rotational arc radiotherapy, and improving the radiotherapy efficiency.

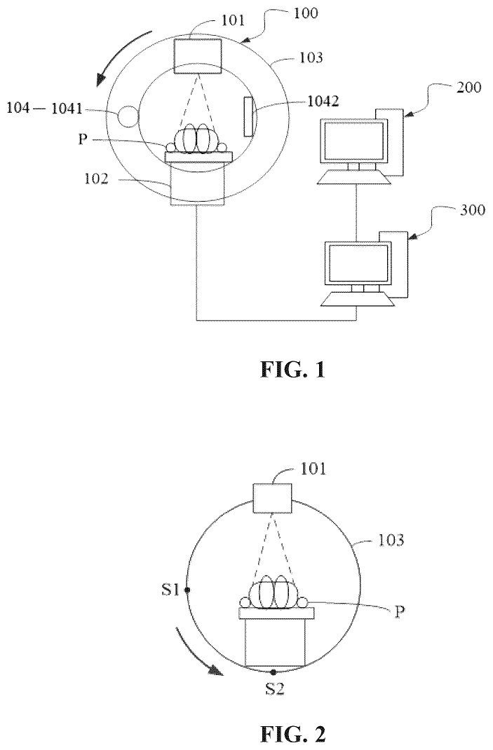

FIG. 1 is a schematic diagram of a radiotherapy system according to some embodiments of the present disclosure. The radiotherapy system is configured to deliver precise radiotherapy to a tumor in the patient's body. The radiotherapy system includes a radiotherapy device 100, a treatment planning system 200, and a control system 300.

The radiotherapy device 100 includes a radiation source 101 (also called a treatment head) capable of emitting therapeutic rays and a treatment couch 102 configured to carry a patient P.

The radiation source 101 may be a radiation source capable of emitting photon rays, for example, a gamma radiation source capable of emitting gamma rays, a medical linear accelerator capable of emitting high-energy X-rays or electron beams, and the like. In some embodiments, the radiation source 101 may also be a radiation source capable of emitting particle rays, for example, a radiation source capable of emitting proton rays, heavy ion rays, and neutron rays.

The treatment couch 102 is configured to carry the patient P, and is capable of driving the patient P to move in a three-dimensional direction (i.e., transverse, longitudinal, and lifting directions) or a six-dimensional direction (i.e., a linear movement in transverse, longitudinal, and lifting directions, and a superimposed rotational movement in transverse, longitudinal, and lifting directions).

The radiation source 101 is capable of rotating around a longitudinal direction of the patient P to enable radiation delivery to the patient P. In order to realize the rotation of the radiation source 101 around the longitudinal direction of the patient P, in some embodiments, the radiotherapy device 100 further includes a gantry 103 configured to carry the radiation source 101, and the radiation source 101 is arranged on the gantry 103. Exemplarily, the gantry 103 may be a ring-shaped rotating gantry or a C-arm rotating gantry. That is, the rotating gantry may rotate around the longitudinal direction of the patient P, such that the radiation source 101 is driven to rotate around the longitudinal direction of the patient. In addition, the gantry 103 may also be a fixed gantry, and the radiation source 101 is configured to move along the gantry 103 around the longitudinal rotation of the patient, i.e., the gantry 103 remains stationary and the radiation source 101 is supported by the gantry 103 to rotate around the longitudinal direction of the patient P.

In some embodiments, when the gantry 103 is a ring-shaped rotating gantry, the treatment couch 102 can move into the ring-shaped rotating gantry along the longitudinal direction of the patient P, and is used for moving the patient P into the ring-shaped rotating gantry to receive the radiation delivered by the radiation source 101.

The treatment planning system 200 is configured to receive a medical image of the patient and develop a treatment plan based on the medical image of the patient for the delivery of a desired radiation dose to the patient.

In the embodiments of the present disclosure, the medical image of the patient refers to a medical image that contains a tumor and surrounding normal tissues and is generated by scanning an affected part of the patient with an image acquisition device (not shown), and the medical image of the patient is uploaded to a treatment planning system. In some embodiments, the image acquisition device may be at least one of a computed tomography (CT) device, an emission computed tomography (ECT) device, a magnetic resonance imaging (MRI) device, a positron emission tomography (PET) device, an ultrasonography device, and the like.

After the treatment planning system 200 receives the medical image of the patient, a physician or physicist sets a dose constraint for a tumor area and surrounding normal tissues according to the patient's medical image. The treatment planning system 200 generates a patient-specific treatment plan based on the medical image of the patient and the dose constraint, and uploads the treatment plan to the control system 300. The treatment plan includes an irradiation direction of the therapeutic rays emitted from the radiation source, an irradiation duration, a dose rate of the therapeutic rays, and the like.

After receiving the treatment plan, the control system 300 analyzes the treatment plan, converts the content of the treatment plan into a control language and/or control parameters that the control system 300 can recognize, and controls the radiotherapy device 100 to perform radiation delivery to the patient P according to the control language and/or control parameters.

Exemplarily, the control system 300 is configured to control a coordinated movement of the radiation source 101, the gantry 103, and the treatment couch 102 to realize the radiation delivery to the patient P. The control system 300 includes a general-purpose computer device or a special-purpose computer device. In a specific implementation, the computer device may be a desktop computer, a portable computer, a network server, a personal digital assistant (PDA), a mobile phone, a tablet computer, a wireless terminal device, a communication device, an embedded device, and the like. The type of the computer device is not limited in this embodiment. The computer device is a computer device with a graphical user interface (GUI), including one or more processors, a memory, and one or more applications. The processors are configured to control the movement of the radiation source, the gantry, and the treatment couch.

In the embodiments of the present disclosure, the processor is configured to control the radiation source to continuously rotate for a radian of greater than 360 degrees in a first direction from a first position around the longitudinal direction of the patient to a second position.

The first position may be anywhere within a 360-degree range of rotation around the longitudinal direction of the patient, and the first direction is a counterclockwise or clockwise direction.

FIG. 2 is a schematic diagram of an embodiment in which a processor of a radiotherapy system controls a radiation source to move according to some embodiments of the present disclosure. As shown in FIG. 2, taking a continuous arc radiotherapy irradiation range of 450 degrees of the treatment plan formulated by the treatment planning system as an example, in order to implement the radiation delivery of the treatment plan, a processor of the radiotherapy system provided by the embodiments of the present disclosure controls the radiation source 101 to rotate in the counterclockwise direction for 360 degrees from a first position S1 around the longitudinal direction of the patient P back to the first position S1, and then continues to control the radiation source 101 to rotate in the counterclockwise direction around the longitudinal direction of the patient P to a second position S2. In addition, the radiation source 101 continues to emit beams to complete the radiation delivery of the entire treatment plan as the radiation source 101 rotates. It is not necessary to allow the radiation source 101 to rotate for 360 degrees back to the first position S1, idle (i.e., the radiation source 101 stops emitting beams) reversely (i.e., in the clockwise direction) to the second position S2, and then continue to rotate in the clockwise direction from the second position S2 to the first position S1 to complete the radiation delivery of the entire treatment plan.

The processor of the radiotherapy system provided by the embodiments of the present disclosure is capable of controlling the radiation source to continuously rotate in the same direction for a radian of more than 360 degrees around the longitudinal direction of the patient. Therefore, in the case of radiation delivery with arc radiotherapy irradiation for greater than 360 degrees, the radiation source does not need to be reversed to complete the radiation delivery, achieving high radiotherapy efficiency.

In some embodiments, the processor of the radiotherapy system provided by the embodiments of the present disclosure is further configured to: continue to control the radiation source to continuously rotate in the first direction from the second position around the longitudinal direction of the patient to the first position. That is, the processor is capable of controlling the radiation source to continuously rotate in the same direction for a radian of 720 degrees around the longitudinal direction of the patient, without the need for the reverse rotation of the radiation source.

In some embodiments, the processor of the radiotherapy system provided by the embodiments of the present disclosure is further configured to: continue to control the radiation source to continuously rotate for any radian in the first direction from the first position around the longitudinal direction of the patient and then stop moving.

Specifically, the processor of the radiotherapy system is further capable of controlling the radiation source to rotate for 720 degrees around the longitudinal direction of the patient, then to continuously rotate for any radian in the first direction around the longitudinal direction of the patient, and then stop moving. For example, the radiation source continues to rotate in the first direction around the longitudinal direction of the patient for 90 degrees, 180 degrees, 360 degrees, 720 degrees, and the like, and then stops moving. That is, the processor is capable of controlling the radiation source to infinitely and continuously rotate in the same direction around the longitudinal direction of the patient.

In some embodiments, the processor is further configured to control the radiation source to deliver a radiation to the patient during the continuous rotational movement of the radiation source around the longitudinal direction of the patient.

Exemplarily, the processor may control the radiation source to deliver a radiation to the patient during the continuous rotational movement of the radiation source around the longitudinal direction of the patient according to the requirements of the treatment plan. In some embodiments, when an irradiation range of the radiation source specified in the treatment plan includes a plurality of irradiation arc segments, the processor may control the radiation source to emit therapeutic rays to the patient at the arc segment that needs to be irradiated by the radiation source for radiation delivery; and control the radiation source to stop irradiating in the are segment that does not need to be irradiated by the radiation source. In some embodiments, when the radiation range specified in the treatment plan is a continuous full circle (e.g., one circle, two cycles, or N circles), the processor may control the radiation source to emit radiation rays to the patient for radiation delivery while continuously rotating around the longitudinal direction of the patient.

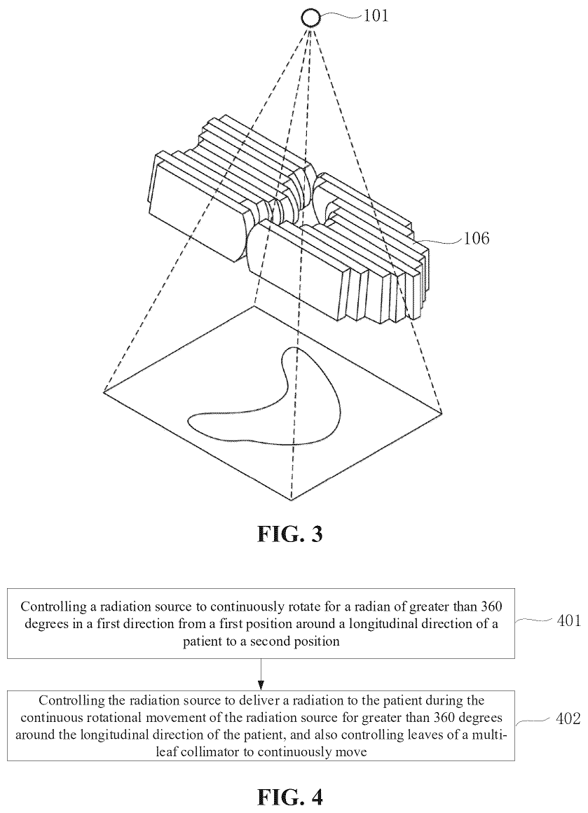

In some embodiments, referring to FIG. 3, FIG. 3 is a schematic structural diagram of a radiation source and a multi-leaf collimator in a radiotherapy system according to some embodiments of the present disclosure. A radiotherapy device of the radiotherapy system provided by the embodiments of the present disclosure further includes a multi-leaf collimator 106. The multi-leaf collimator 106 is configured to perform beam shaping on the therapeutic rays emitted from the radiation source. The processor provided by the embodiments of the present disclosure is further configured to control the continuous movement of leaves of the multi-leaf collimator while the radiation source delivers the radiation to the patient.

Referring to FIG. 3, the multi-leaf collimator 106 includes a plurality of leaf pairs arranged in a width direction of the leaves, each of which is capable of independently and continuously moving in a length direction of the leaf (e.g., each leaf may move continuously to approach a radiation beam). Through the independent and continuous movement of the leaves in the plurality of leaf pairs, an aperture is formed to match the shape of the tumor, As the leaves of the multi-leaf collimator are made of a material (e.g., lead, or tungsten) that shields the therapeutic rays, the multi-leaf collimator can shield the therapeutic rays outside the aperture and deliver the radiation to the tumor area by using the therapeutic rays that pass through the aperture, so as to achieve the beam shaping of the therapeutic rays.

In order to obtain a higher radiation dose to the tumor area while allowing the surrounding normal tissues to receive a minimum radiation dose, the processor provided by the embodiments of the present disclosure is further configured to: control the continuous movement of the leaves of the multi-leaf collimator while the radiation source delivers the radiation to the patient, that is, the leaves of the multi-leaf collimator also move continuously as the radiation source emits beams.

In some examples, the processor provided by the embodiments of the present disclosure is further configured to control the treatment couch to remain stationary during the delivery of the radiation from the radiation source to the patient. That is, the processor controls the treatment couch to remain stationary and not move when the radiation source emits beams.

In some examples, the processor provided by the embodiments of the present disclosure is further configured to control the treatment couch to move in the longitudinal direction of the patient during the delivery of the radiation from the radiation source to the patient. Specifically, the processor controls the treatment couch to move unidirectionally at a uniform or non-uniform speed or reciprocate in the longitudinal direction of the patient when the radiation source emits beams.

In some embodiments, the radiotherapy device of the radiotherapy system provided by the embodiments of the present disclosure further includes an imaging apparatus 104. The imaging apparatus 104 is configured to acquire an image of the patient. The processor provided by the embodiments of the present disclosure is further configured to control the imaging apparatus to acquire the image of the patient during the continuous rotational movement of the radiation source around the longitudinal direction of the patient.

Referring to FIG. 1, the imaging apparatus 104 includes an imaging source 1041 and a detector 1042 arranged oppositely. Imaging rays emitted from the imaging source 1041 are received by the detector 1042 after passing through the patient's tumor site and are used for generating an image of the patient's tumor site. In some embodiments, the processor controls the imaging apparatus 104 to acquire the image of the patient during the continuous rotational movement of the radiation source around the longitudinal direction of the patient. When the imaging apparatus 104 acquires the image of the patient, the radiation source 101 may be in a state of emitting beams or in a state of stopping emitting beams. That is, the image acquisition of the patient may be performed simultaneously or alternately with the beam emission of the radiation source.

In some embodiments, the radiotherapy apparatus of the radiotherapy system according to the embodiments of the present disclosure further includes a slip ring for realizing infinitely continuous rotation of the radiation source. The slip ring includes a stator and a rotor. The stator and the rotor are communicated through a conductive loop. The transmission of power, signals, and the like of the radiation source is realized through the relative rotation between the stator and the rotor. Exemplarily, a stator lead wire of the slip ring is connected to a power source/signal source, and a rotor lead wire is connected to the radiation source. Through the rotational movement of the radiation source, the stator and the rotor come into rotary contact to provide power/signals to the radiation source.

In some embodiments, the radiotherapy device may also use other means (e.g., a towing chain) to realize the continuous rotation of the radiation source for a radian of greater than 360 degrees around the longitudinal direction of the patient. An implementation mode of the continuous rotation of the radiation source for a radian of greater than 360 degrees around the longitudinal direction of the patient is not limited in the present disclosure.

The processor of the radiotherapy system provided by the embodiments of the present disclosure is capable of controlling the radiation source to continuously rotate in the same direction for a radian of more than 360 degrees around the longitudinal direction of the patient. Therefore, in the case of radiation delivery with arc radiotherapy irradiation for greater than 360 degrees, the radiation source does not need to rotate reversely to complete the radiation delivery, achieving high radiotherapy efficiency.

The present disclosure further provides a method for controlling a radiotherapy system, which is applied to a processor of the radiotherapy system according to the above embodiments. The method includes: controlling the radiation source to continuously rotate for a radian of greater than 360 degrees in a first direction from a first position around a longitudinal direction of the patient to a second position,

Therefore, according to the method for controlling the radiotherapy system provided by the embodiments of the present disclosure, the radiation source can be controlled to continuously rotate in the same direction for a radian of more than 360 degrees around the longitudinal direction of the patient. Therefore, in the case of radiation delivery with arc radiotherapy irradiation for greater than 360 degrees, the radiation source does not need to be rotated reversely to complete the radiation delivery, achieving high radiotherapy efficiency.

The present disclosure further provides a method for controlling a radiotherapy system, which is applied to a processor of the radiotherapy system according to the above embodiments. As shown in FIG. 4, FIG. 4 is a flowchart of a method for controlling a radiotherapy system according to some embodiments of the present disclosure. The method includes the following steps.

In step 401, a radiation source is controlled to continuously rotate for a radian of greater than 360 degrees in a first direction from a first position around a longitudinal direction of a patient to a second position.

In step 402, the radiation source is controlled to deliver a radiation to the patient during the continuous rotational movement of the radiation source for greater than 360 degrees around the longitudinal direction of the patient, and leaves of a multi-leaf collimator are also controlled to continuously move.

Therefore, according to the method for controlling the radiotherapy system provided by the embodiments of the present disclosure, the radiation source can be controlled to continuously rotate in the same direction for a radian of more than 360 degrees around the longitudinal direction of the patient, and therapeutic rays are subjected to beam shaping dynamically at the same time to modulate a shape and dose of beams. Therefore, in the case of radiation delivery with arc radiotherapy irradiation for greater than 360 degrees, the radiation source does not need to be reversed while ensuring that the delivered radiation is modulated, thereby improving the efficiency and precision of the radiotherapy.

The radiotherapy system and the method for controlling the same provided by the embodiments of the present disclosure are introduced above in detail. Specific examples are used herein to illustrate the principles and embodiments of the present disclosure. The description of the above embodiments is only used to help the understanding of the methods and core ideas of the present disclosure. At the same time, for those skills in the art, according to the ideas of the present disclosure, there will be changes in the specific embodiments and the scope of application. In summary, the content of the present description should not be construed as a limitation of the present disclosure.

1. A radiotherapy system, comprising:
a radiation source configured to emit therapeutic rays, wherein the radiation source is capable of rotating around a longitudinal direction of a patient;
a treatment couch configured to carry the patient; and
a processor configured to control the radiation source to continuously rotate for a radian of greater than 360 degrees in a first direction from a first position around the longitudinal direction of the patient to a second position.

2. The radiotherapy system according to claim 1, wherein the processor is further configured to continue to control the radiation source to continuously rotate in the first direction from the second position around the longitudinal direction of the patient to the first position.

3. The radiotherapy system according to claim 2, wherein the processor is further configured to continue to control the radiation source to continuously rotate for any radian in the first direction from the first position around the longitudinal direction of the patient and then stop moving.

4. The radiotherapy system according to claim 1, wherein the processor is further configured to control, during a continuous rotational movement of the radiation source around the longitudinal direction of the patient, the radiation source to deliver a radiation to the patient.

5. The radiotherapy system according to claim 4, further comprising a multi-leaf collimator, wherein the multi-leaf collimator is configured to perform beam shaping on the therapeutic rays emitted from the radiation source; and the processor is further configured to control continuous movement of leaves of the multi-leaf collimator while the radiation source delivers the radiation to the patient.

6. The radiotherapy system according to claim 4, wherein the processor is further configured to control, during a delivery of the radiation from the radiation source to the patient, the treatment couch to remain stationary.

7. The radiotherapy system according to claim 4, wherein the processor is further configured to control, during a delivery of the radiation from the radiation source to the patient, the treatment couch to move in the longitudinal direction of the patient.

8. The radiotherapy system according to claim 1, further comprising an imaging apparatus, wherein the imaging apparatus is configured to acquire an image of the patient; and the processor is further configured to control, during a continuous rotational movement of the radiation source around the longitudinal direction of the patient, the imaging apparatus to acquire the image of the patient.

9. The radiotherapy system according to claim 1, further comprising a rotating gantry, wherein the radiation source is arranged on the rotating gantry; and the radiation source rotates around the longitudinal direction of the patient under a drive of the rotating gantry.

10. The radiotherapy system according to claim 9, wherein the rotating gantry is a ring-shaped gantry; and the treatment couch is capable of moving into the ring-shaped gantry along the longitudinal direction of the patient.

11. A radiotherapy system, comprising:
a radiation source configured to emit therapeutic rays, wherein the radiation source is capable of rotating around a longitudinal direction of a patient;
a treatment couch configured to carry the patient;
a multi-leaf collimator configured to perform beam shaping on the therapeutic rays emitted from the radiation source; and
a processor configured to control the radiation source to continuously rotate for a radian of greater than 360 degrees in a first direction from a first position around the longitudinal direction of the patient to a second position; and control the radiation source to deliver a radiation to the patient during a continuous rotational movement of the radiation source for greater than 360 degrees around the longitudinal direction of the patient, and also control leaves of the multi-leaf collimator to continuously move.

12. The radiotherapy system according to claim 11, wherein the processor is further configured to continue to control the radiation source to continuously rotate in the first direction from the second position around the longitudinal direction of the patient to the first position.

13. The radiotherapy system according to claim 12, wherein the processor is further configured to continue to control the radiation source to continuously rotate for any radian in the first direction from the first position around the longitudinal direction of the patient and then stop moving.

14. The radiotherapy system according to claim 11, wherein the processor is further configured to control, during a delivery of the radiation from the radiation source to the patient, the treatment couch to remain stationary.

15. The radiotherapy system according to claim 11, wherein the processor is further configured to control, during a delivery of the radiation from the radiation source to the patient, the treatment couch to move in the longitudinal direction of the patient.

16. The radiotherapy system according to claim 11, further comprising an imaging apparatus, wherein the imaging apparatus is configured to acquire an image of the patient; and the processor is further configured to control, during a continuous rotational movement of the radiation source around the longitudinal direction of the patient, the imaging apparatus to acquire the image of the patient.

17. The radiotherapy system according to claim 11, further comprising a rotating gantry, wherein the radiation source is arranged on the rotating gantry; and the radiation source rotates around the longitudinal direction of the patient under a drive of the rotating gantry.

18. The radiotherapy system according to claim 17, wherein the rotating gantry is a ring-shaped gantry; and the treatment couch is capable of moving into the ring-shaped gantry in the longitudinal direction of the patient.

19. A method for controlling a radiotherapy system, applied to a processor of the radiotherapy system as defined in claim 1, and comprising:
controlling the radiation source to continuously rotate for the radian of greater than 360 degrees in the first direction from the first position around the longitudinal direction of the patient to the second position.

20. A method for controlling a radiotherapy system, applied to a processor of the radiotherapy system, wherein the radiotherapy system comprises: a radiation source configured to emit therapeutic rays; a treatment couch configured to carry a patient; the radiation source being capable of rotating around a longitudinal direction of the patient; a multi-leaf collimator configured to perform beam shaping on the therapeutic rays emitted from the radiation source; and a processor configured to control the radiation source and the multi-leaf collimator; and the method comprises:
controlling the radiation source to continuously rotate for a radian of greater than 360 degrees in a first direction from a first position around the longitudinal direction of the patient to a second position; and
controlling the radiation source to deliver a radiation to the patient during a continuous rotational movement of the radiation source for greater than 360 degrees around the longitudinal direction of the patient, and also controlling leaves of the multi-leaf collimator to continuously move.
